(19) United States
(12) Patent Application Publication
(10) Pub. No.: US 2025/0345408 A1
(43) Pub. Date: Nov. 13, 2025

(54) BCG VACCINATIONS FOR PREVENTION OF COVID-19 AND OTHER INFECTIOUS DISEASES

(71) Applicant: The General Hospital Corporation, Boston, MA (US)

(72) Inventor: Denise L. FAUSTMAN, Boston, MA (US)

(21) Appl. No.: 18/027,696

(22) PCT Filed: Sep. 23, 2021
(86) PCT No.: PCT/US2021/051775

§ 371 Date: Mar. 22, 2023

Related U.S. Application Data
(60) Provisional application No. 63/082,094, filed on Sep. 23, 2020.

(51) Int. Cl. A61K 39/04; A61K 39/00; A61P 37/04

(52) U.S. Cl. CPC A61K 39/04; A61P 37/04; A61K 2039/522; A61K 2039/545; A61K 2039/58

(57) ABSTRACT

The invention relates, in part, to a method for the prophylactic treatment of a coronavirus infection in a human adult subject comprising administering at least two doses of a Bacillus Calmette-Guerin (BCG) vaccine to the subject, wherein the subject is a type I diabetic.

### CROSS-REFERENCE TO RELATED APPLICATIONS

This application claims benefit of U.S. Provisional Application No. 63/082,094, filed on Sep. 23, 2020, the contents of which are incorporated herein by reference in their entirety.

### BACKGROUND OF THE INVENTION

As the Covid-19 pandemic worsens and threats of new pandemics are ever present, vaccine development assumes center stage. But antigen-focused vaccines are struggling to keep pace with new viral variants. The ideal vaccine should be safe, efficacious, affordable, and offer durable protection against ever-changing viral variants and future pandemics. There is, accordingly, a need for safe and effective platform vaccines to protect against Covid-19 and other infectious diseases.

### SUMMARY OF THE INVENTION

In one aspect, the invention features, in general, a method for the prophylactic treatment of a coronavirus infection in a subject which includes administering at least two doses of a Bacillus Calmette-Guerin (BCG) vaccine.

In another aspect, the invention features a method for the prophylactic treatment of a coronavirus infection in a subject which includes administering at least two doses of a BCG vaccine to the subject, wherein the subject has not been previously vaccinated with a BCG vaccine.

In still other aspects, the invention features a method for the prophylactic treatment of a coronavirus infection in a human adult subject which includes administering at least two doses of a BCG vaccine to the subject, wherein the subject has a co-morbidity selected from obesity, diabetes (Type I or Type II), hypertension, hypercholesteremia, cancer, chronic kidney disease, COPD, coronary vascular disease, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19.

In any one of the aforementioned aspects, the coronavirus is SARS COV-2.

In some embodiments, the subject typically has not been previously vaccinated with a BCG vaccine.

In other embodiments, the volume of the dose is delivered as about 0.1 ml volume and the BCG is about 2.09 to 50×10⁸ cfu per 0.5 mg BCG/10 doses. The dose is usually administered intradermally or percutaneously.

In other aspects, the method for the prophylactic treatment of a SARS COV-2 virus infection in a subject includes administering at least two doses of a BCG vaccine to the subject, wherein the subject has a co-morbidity. In some embodiments, the co-morbidity is selected from obesity, diabetes (Type I or Type II), hypertension, hypercholesteremia, cancer, chronic kidney disease, COPD, coronary vascular disease, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19.

In embodiments of the various aspect, the subject is age 18 or older; the subject is age 12 to 17;

or the subject is 11 months to age 11.

In some embodiments, the subject received three doses of BCG vaccine.

In other embodiments, the subject received greater than three doses of BCG vaccine.

In other embodiments, the subject received BCG (Tokyo-172 strain) vaccine.

In general, the subject may receive two doses of BCG vaccine four weeks apart.

In one embodiment, the subject is a Type I diabetic receiving repeat BCG vaccinations (e.g., the Type I diabetic receives two doses of BCG vaccine four weeks apart). In other embodiments, the subject is an established Type I diabetic.

Subjects typically receive a booster vaccine dose.

Subjects may also have one or more co-morbidities. For example, the subject may be a type 1 diabetic having hypercholesteremia.

In other aspect, the invention features a method for the prophylactic treatment of a viral infection in a subject which includes administering at least two doses of a BCG vaccine to the patient not at the time of birth. Such a viral infection is due to, for example, a coronavirus, rhinovirus, coxsackie virus, enterovirus or polio virus.

Accordingly, in yet another aspect the invention includes a method for the prophylactic treatment of a viral infection in a human adult patient including administering at least two doses of a BCG vaccine to the patient, wherein the patient has not been previously vaccinated with a BCG vaccine or wherein the patient has a co-morbidity selected from obesity, diabetes (Type I or Type II), hypertension, hypercholesteremia, cancer, chronic kidney disease, chronic obstructive pulmonary disease (COPD), coronary vascular disease, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19.

The virus may be any coronavirus, particularly a SARS COV-2 coronavirus, but the method of the invention is applicable to infections caused by these viruses as well: rhinovirus, Coxsackie virus, Torque Teno virus, polio virus, enterovirus, echovirus, papilloma virus, adenovirus, hepatitis virus (A, B, C, E), herpes simplex virus, Epstein Barr virus, influenza virus, parainfluenza virus, respiratory syncytial virus, cytomegalovirus, small pox virus, rabies virus, ebola virus, hanta fever virus, vaccinia virus, powassan virus, mamastrovirus, astrovirus, New York virus, Rift Valley Fever virus, Southampton, Sapporovirus, Sandfly Fever virus, Madariaga virus, Dengue virus, Orf virus, adeno-associated virus, Bunyamwere virus, Seoul virus, human immunodeficiency virus, Hantaan virus, KI polyomavirus, Lake Victoria marburg virus, hemagglutinating encephalomyelitis virus, Bunyavirus, cosavirus, Chandipura virus, lymphocytic choriomeningitis virus, Dhori virus, Simian foamy virus, Duvenhage virus, O'nyong-nyong virus, Oropouche virus, Cowpox virus, hepatitis delta virus, Lassa virus, Banna virus, St. Louis encephalitis virus, vesicular stomatitis virus, West Nile virus, Yellow Fever virus, rotavirus, aichi virus, encephalomyocarditis virus, Sandfly fever virus, Machupo virus, Zika virus, hemorrhagic fever virus, BK polyoma virus, Puumala virus, Kunjin virus, Mokola virus, rubla virus, and varicella zoster virus.

In another aspect, the invention includes a method for the prophylactic treatment of a coronavirus infection in a human adult patient including administering at least two doses of a BCG vaccine to the patient, wherein the patient has not been previously vaccinated with a BCG vaccine and wherein the patient has a co-morbidity selected from obesity, diabetes (Type I or Type II), hypertension, cancer, chronic kidney disease, COPD, coronary vascular disease, hypercholesteremia, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19. Adult patients are those over the age of 18.

The BCG vaccine may be lyophilized and reconstituted before administration and the administration can be intradermal or percutaneous. Any skin site on the body can be used but traditionally the vaccine is administered in the upper arms. The multiple dosages could be administered simultaneously at different sites or weeks apart in order to facilitate the systemic protection. Repeat yearly doses in adults of up to six (6) vaccines have proven safe and effective in clinical trials conducted at the MGH in adult patients with co-morbid conditions. Each dose can conform to the vaccine doses and typically involve dosing described as live bacteria of BCG with approximately 50-90% moist bacteria with a stabilizer such as sodium glutamate. The volume of the dose is typically 0.1 ml and the BCG should be 2.09 to 50×10⁸ cfu per 0.5 mg BCG. This typically will yield from 10-30×10⁶ cfu.

The invention further includes the following embodiments according to the following numbered paragraphs.
1. A method for the prophylactic treatment of a viral infection in a human adult patient comprising administering at least two doses of a BCG vaccine to the patient not at the time of birth.
2. The method according to paragraph 1 wherein the viral infection is due to a coronavirus, rhinovirus, coxsackie virus, enterovirus or polio virus.
3. A method for the prophylactic treatment of a coronavirus infection in a human adult patient comprising administering at least two doses of a BCG vaccine to the patient not at the time of birth.
4. A method for the prophylactic treatment of a coronavirus infection in a human adult patient comprising administering at least two doses of a BCG vaccine to the patient, wherein the patient has not been previously vaccinated with a BCG vaccine.
5. A method for the prophylactic treatment of a coronavirus infection in a human adult patient comprising administering at least two doses of a BCG vaccine to the patient, wherein the patient has a co-morbidity selected from obesity, diabetes (Type I or Type II), hypertension, hypercholesteremia, cancer, chronic kidney disease, COPD, coronary vascular disease, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19.
6. The method according to any one of paragraphs 3-5 wherein the coronavirus is SARS CoV-2.
7. The method according to any one of paragraphs 1-3 wherein the patient has not been previously vaccinated with a BCG vaccine.
8. The method according to any one of paragraphs 1-7 wherein the volume of the dose is delivered as about a 0.1 ml volume and the BCG is about 2.09 to 50×10⁶ cfu per 0.5 mg BCG/10 doses.
9. The method according to any one of paragraphs 1-8 wherein the dose is administered intradermally or percutaneously.
10. A method for the prophylactic treatment of a SARS COV-2 virus infection in a human adult patient comprising administering at least two doses of a BCG vaccine to the patient, wherein the patient has a co-morbidity and wherein the patient has not been previously vaccinated with a BCG vaccine.
11. The method according to paragraph 10 wherein the co-morbidity is selected from obesity, diabetes (Type I or Type II), hypertension, hypercholesteremia, cancer, chronic kidney disease, COPD, coronary vascular disease, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19.

Further embodiments include the following numbered paragraphs.
1. A method for the prophylactic treatment of a coronavirus infection in a subject comprising administering at least two doses of a BCG vaccine to the subject not at the time of birth.
2. A method for the prophylactic treatment of a coronavirus infection in a subject comprising administering at least two doses of a BCG vaccine to the subject, wherein the subject has not been previously vaccinated with a BCG vaccine.
3. A method for the prophylactic treatment of a coronavirus infection in a human adult subject comprising administering at least two doses of a BCG vaccine to the subject, wherein the subject has a co-morbidity selected from obesity, diabetes (Type I or Type II), hypertension, hypercholesteremia, cancer, chronic kidney disease, COPD, coronary vascular disease, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19.
4. The method according to any one of paragraphs 1-3, wherein the coronavirus is SARS CoV-2.
5. The method according to any one of paragraphs 1 and 3, wherein the subject has not been previously vaccinated with a BCG vaccine.
6. The method according to any one of paragraphs 1-5, wherein the volume of the dose is delivered as about a 0.1 ml volume and the BCG is about 2.09 to 50×10⁶ cfu per 0.5 mg BCG/10 doses.
7. The method according to any one of paragraphs 1-6 wherein the dose is administered intradermally or percutaneously.
8. A method for the prophylactic treatment of a SARS COV-2 virus infection in a subject comprising administering at least two doses of a BCG vaccine to the subject, wherein the subject has a co-morbidity and wherein the subject has not been previously vaccinated with a BCG vaccine.
9. The method according to paragraph 8 wherein the co-morbidity is selected from obesity, diabetes (Type I or Type II), hypertension, hypercholesteremia, cancer, chronic kidney disease, COPD, coronary vascular disease, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19.
10. The method according to any one of paragraphs 1-9, wherein the subject is age 18 or older.
11. The method according to any one of paragraphs 1-9, wherein the subject is age 12 to 17.
12. The method according to any one of paragraphs 1-9, wherein the subject is 11 months to age 11.
13. The method according to any one of paragraphs 1-12, wherein the subject received three doses of BCG vaccine.
14. The method according to any one of paragraphs 1-12, wherein the subject received greater than three doses of BCG vaccine.
15. The method according to any one of paragraphs 1-14, wherein the subject received BCG (Tokyo-172 strain) vaccine.
16. The method according to any one of paragraphs 1-15, wherein the subject is a Type I diabetic receiving repeat BCG vaccinations.
17. The method according to any one of paragraphs 1-12, wherein the subject receives two doses of BCG vaccine four weeks apart.
18. The method according to claim 17, wherein the subject receives a booster vaccine dose.
19. The method according to any one of paragraphs 1-18, wherein the subject is an established Type 1 diabetic.
20. The method of paragraph 17, wherein the Type I diabetic had at least one co-morbidity.
21. A method for the prophylactic treatment of a viral infection in a subject comprising administering at least two doses of a BCG vaccine to the patient not at the time of birth.
22. The method according to paragraph 21, wherein the viral infection is due to a coronavirus, rhinovirus, coxsackie virus, enterovirus or polio virus.

The invention provides numerous advantages. For example, the BCG vaccine, disclosed herein, effectively protects against Covid-19 and is safe, effective, affordable, and is likely protective against new variants based on its broad-based protection against other infections. To date, in the disclosed methods, the known safe BCG vaccine had no unexpected side effects or symptoms at the time of vaccination.

The COVID-19 pandemic triggered the pursuit of antigen vaccines, but the SARS-COV-2 virus continues to mutate. Expensive vaccine development may not continue to be feasible for the current and future pandemics and a platform vaccine strategy is needed. The methods disclosed herein satisfy such a need.

The methods disclosed herein can protect from both COVID-19 and a variety of infections. Advantageously, the incidence of symptomatic COVID-19 disease was lower among BCG recipients than placebo recipients. Still further, the overall incidence of infection-related adverse events was also reduced in the BCG-vaccinated subjects. BCG vaccinations additionally provided 92% efficacy against symptomatic COVID-19 and offered broad-based resistance to symptomatic infections.

Other features and advantages of the invention will be apparent from the following description of the preferred embodiments thereof, and from the claims.

### BRIEF DESCRIPTION OF THE FIGURES

FIGS. 1A-1B show enrollment and randomization of participants. FIG. 1A shows a consort diagram representing all enrolled participants from Jan. 1, 2020 to April 2021 for this double-blinded randomized clinical trial testing repeat Tokyo-172 BCG vaccination vs placebo. FIG. 1B shows that all 144 participants were US citizens not previously vaccinated with BCG at birth and had long-standing type 1 diabetes, a co-morbid condition for worse Covid-19 symptoms.

FIGS. 2A-2B show BCG vaccine efficacy and diagnostic confirmation of Covid-19 disease. FIG. 2A shows the cumulative incidence of confirmed Covid-19 as a primary endpoint of a 15-month clinical trial. Vaccine efficacy was defined as (p1−p2)/p1×100 where p1 is the percentage Covid-positive subjects in the placebo group (12.5%) and p2 is the percentage Covid-positive subjects in the BCG group (1%). The criteria for confirmed Covid-19 were as follows: reporting symptom(s) of Covid-19, as well as testing positive for 5 or more out of 10 in vitro diagnostic tests. Cumulative findings from these 10 diagnostic tests are shown in FIG. 2B. During the 15-month surveillance time, one BCG recipient out of 96 (1.0%) met our criteria for confirmed Covid-19. In contrast, 6 out of 48 placebo recipients met the criteria (12.5%). Fisher's Exact testing showed a significant difference (two-tailed p=0.006). Calculated vaccine efficacy was 92% and the posterior probability (vaccine efficacy >30%) was 0.99. This was calculated using the Monte Carlo method. The diagnostic test details are represented in Table 1. FIG. 2B shows 10 in-vitro diagnostic tests used to diagnose Covid-19 (along with positive symptoms). These included the presence of Covid-19-specific antibodies to various SARS-COV-2 virus epitopes through protein display (I-VIII), antibodies to the Spike protein with an ELISA test (IX) and Point of Care testing (X). Thus the trial required at least 5 of 10 detection methods to be positive, along with symptoms, for having confirmed Covid-19. For the antibody assays, a patient was considered positive when the test resulted in a Z-score of ≥3. In the cumulative graphs, the x-axis data shows the time-period of the 15-month trial. The Y-axis shows the cumulative percentage of positive subjects. Except for the point of care graph (X), all other graphs represent the percentage of BCG and placebo patients with a Z-score of ≥3 for the anti-SARS-CoV-2 antibody binding to a given protein region of the virus, i.e. the average antibody level during the Covid trial period was at least 3 standard deviations greater than the average level in the period preceding the Covid trial (baseline). The percentiles at the top-right of each graph represent the calculated vaccine efficacy if this test alone were used to diagnose Covid-19 disease. Respective efficacy and Fisher's Exact p values for each Covid-19 antibody test were as follows: (I) 100%, p=0007; (II) 80%, p=0.089; (III) 91.7%, p=0.009; (IV) 93.8%, p=0.001; (V) 90%, p=0.024; (VI) 87.5%, p=0.004; (VII) 94.4%, p=0.0003; (VIII) 83.3%, p=0.009, (IX) 91.7%, p=0.029; (X) 90%, p=0.009; Table 1 shows the viral protein regions for each anti-Covid antibody tested.

FIGS. 3A-3B show cumulative infections. FIG. 3A shows cumulative total infectious diseases during the 15 months of surveillance. This cumulative figure shows all infections per patient, including all Covid-19 events, within the BCG group compared to the placebo group (red). Included are the infections for which multiple events were documented in both BCG and placebo groups. Comparison by means of a Poisson model yields a significant difference with p=0.004. See also Table 3. FIG. 3B shows cumulative infectious diseases for two different time periods: pre-Covid-19 pandemic (the two and one-half year period prior to this trial, i.e., the pre-trial) and during the Covid-19 pandemic (the clinical trial period of 15 months). The pre-trial time period was when all clinical trial subjects received their ≥3 BCG vaccines or placebo vaccines; the Current Trial was when the subjects were monitored during the 15-month observation period of this clinical trial during the Covid-19 pandemic. The lack of a statistical difference in infectious diseases between BCG and placebo groups in the pre-trial period suggests the 2½-year period with the BCG vaccinations underway might not yet fully protect against infectious disease and thus a longer length of time is necessary for maximal infectious disease protection. It appears by the time this parallel clinical trial started the prior BCG vaccinations were protecting from Covid-19 and all infections. **p<0.01.

FIGS. 4A-4C show infection severity. FIG. 4A shows an Infectious Disease Index for symptomatic patients, expressed as Cohort Total Index and Cohort Average Index. The symptomatic patients in the BCG-treated group had significantly reduced Total Infectious Disease Index (placebo 152±70 (n=20) vs. BCG 48±11 (n=31), p=0.04, single tail and unpaired) as well as Average Infectious Disease Index (Placebo 23±7 (n=20) and BCG 13±2 (n=31), p=0.04, single tail and unpaired). We first calculated total and average symptom scores per patient and then calculated average and s.e.m. of each of these for BCG and placebo cohorts separately (*Student's T-test p<0.05, 1-tailed, unpaired). FIG. 4B shows patients in the BCG cohort reported significantly fewer days of missed work as compared to the placebo group as it related to infections (*p=0.02). FIG. 4C shows the average scores for each infectious symptom separately. The placebo group had more severe average symptoms as compared to the BCG group for 12 out of 12 symptoms (Left). For each symptom, the number of patients in BCG and placebo groups that reported symptoms was then expressed as a percentage of patients in each group (Right). For 11 out of 12 symptoms there was a higher percentage of symptomatic patients in the placebo group as compared to the BCG treated group. Statistical analysis by Student's T-test (1-tailed, unpaired, *p<0.05)

FIGS. 5A-5B show infection symptoms of trial participants vs. adult household members. FIG. 5A shows infection symptoms in BCG and placebo clinical trial groups, compared to non-diabetic adult partners living in the same households. We collected the symptoms of infectious diseases from surveys from all trial participants and household members of 13 BCG families and 7 placebo families. The BCG-treated trial participants had comparable or lower Total Infectious Symptoms Indexes as compared to their partners living in the same household, whereas most Placebo-treated trial participants had more severe symptoms as compared to their partners. Statistical analysis of the differences by Two-sample Wilcoxon testing was significant (2-tailed; p=0.049). FIG. 5B shows distribution of individual infectious disease symptoms in BCG and placebo participants and infected household members across the four Scoring possibilities (0 no symptoms; 1 mild; 2 moderate; 3 severe symptoms). Symptom severity (0=none; 1=mild; 2=moderate; 3=severe) and type (12 categories) were used to calculate total score (maximum=36) and average score.

FIG. 6 shows calculation of average and total infectious symptom index.

### DETAILED DESCRIPTION

Described herein are compositions and methods for prophylactic treatment of a viral infection in a human adult subject which includes administering at least two doses of a BCG vaccine to the subject not at the time of birth. In embodiments, the viral infection is due to a coronavirus, rhinovirus, coxsackie virus, enterovirus, or polio virus. In other embodiments, the patient has a co-morbidity such as obesity, diabetes (Type I or Type II), hypertension, hypercholesteremia, cancer, chronic kidney disease, chronic obstruction pulmonary disease (COPD), coronary vascular disease, cardiomyopathy, cerebrovascular disease or other heart disease or condition, cystic fibrosis, sickle cell disease, pulmonary fibrosis, dementia, pregnancy, liver disease, or is immunocompromised, has a respiratory condition due associated with smoking or vaping, or has any other co-morbidity commonly associated with COVID-19. Typically, the coronavirus is SARS COV-2.

### Dosing Regimens and Routes of Administration

### Dosing Schedules for Administration of BCG

In some embodiments of the disclosure, BCG is administered to a subject (e.g., a human subject, such as a human subject having diabetes (e.g., type 1 diabetes or established type 1 diabetes)) in a single dose. Alternatively, BCG may be administered to the subject in multiple doses. For example, using BCG may be administered to the subject in two doses four weeks apart followed by a booster one year later. In some embodiments, such as protection from viruses described herein, the BCG Is administered to the subject in from 1 to 5 doses or more per year (e.g., in 1, 2, 3, 4, or 5 doses per year). In some embodiments, multidosing spanning at least 2 weeks and up to 2 years for maximal efficacy is employed.

Subjects are, in general, human patients. Such subjects may typically have one or more of the co-morbidities described herein. Such subjects are also typically born in the United States. Still other subjects have not received a BCG vaccine at the time of their birth. Still other subjects may have a comorbidity and receive a dosing regiment of BCG as is described herein. Subjects may range in age from 11 months to age 18 or older.

### Routes of Administration

Using the compositions and methods of the disclosure, BCG can be administered to a subject (e.g., a mammalian subject, such as a human) by a variety of routes. For example, BCG may be administered to a subject intradermally, percutaneously, subcutaneously, orally, transdermally, intranasally, intravenously, intramuscularly, intraocularly, parenterally, intrathecally, or intracerebroventricularly (e.g., intradermally or subcutaneously).

### Pharmaceutical Compositions

Therapeutic compositions containing BCG can be prepared, e.g., using methods known in the art or described herein. For instance, BCG formulations can be prepared using physiologically acceptable carriers, excipients, and/or stabilizers (Remington's Pharmaceutical Sciences 16th edition, Osol, A. Ed. (1980); the disclosure of which is incorporated herein by reference), and in a desired form, e.g., in the form of aqueous solutions or suspensions. The compositions can also be prepared to contain BCG at a desired concentration or cell count. BCG compositions of the disclosure also include lyophilized compositions that can be rehydrated prior to administration. The sections that follow describe useful additives that can be included in a BCG formulation for administration to a subject or for long-term storage.

### Strains of BCG

A variety of strains of BCG may be used in conjunction with the compositions and methods of the disclosure. Exemplary strains of BCG include those that can be cultured under good manufacturing protocols, such as the Pasteur, Phipps, Frappier, Mexico, Birkhaug, Sweden, Moreau, Japan-Tokyo, Copenhagen, TICE, Sanofi, Aventis, Connaught, RIVM, Russian, Evans, MMC, Moreau, and Glaxo substrains of BCG, among others, as well as genetic variants of these substrains. Mycobacteria that may be used in conjunction with the compositions and methods of the disclosure may be live, attenuated, or inactivated such that the bacteria retain certain antigen expression patterns but are no longer virulent.

### Uses

In some embodiments, the BCG vaccine and the methods described herein are useful for the prevention or the lessening of symptoms of COVID-19 infections in subjects at higher risk such as autoimmune diseases. In other embodiments, the disclosed compositions and methods prevent or lessen symptoms of infectious diseases in subjects at higher risk such as autoimmune disease (e.g., type 1 diabetes). Still further, the compositions and methods prevent or lessen symptoms of COVID-19 infections in subjects at higher risk such type 1 diabetes. In other embodiments, the compositions and methods prevent or lessen symptoms infectious diseases in subjects at higher risk such as type 1 diabetes.

The BCG vaccine methodology is further useful for the prevention or the lessening of symptoms of infectious diseases not related to tuberculosis. The BCG vaccine is useful for the prevention or the lessening of symptoms of viral infections. The BCG vaccine is still further useful for the prevention or the lessening of symptoms of COVID-19 infections. The BCG vaccine is also useful for the prevention or the lessening of symptoms of infectious diseases in subjects at higher risk and not related to tuberculosis. The BCG vaccine is useful for the prevention or the lessening of symptoms of viral infections in subjects at higher risk. The BCG vaccine is also useful for the prevention or the lessening of symptoms of COVID-19 infections in subjects at higher risk. The BCG vaccine is also useful for the prevention or the lessening of symptoms of infectious diseases in subjects at higher risk. The BCG vaccine still further is useful for the prevention or the lessening of symptoms of viral infections in subjects at higher risk for autoimmune diseases.

As with any vaccine the BCG vaccine may not protect all subjects.

### EXAMPLES

The following examples are put forth to provide those of ordinary skill in the art with a description of how the compositions and methods claimed herein are performed, made, and evaluated, and are intended to be purely exemplary described herein and are not intended to limit the scope of any invention disclosed herein.

### Example 1

Massachusetts General Hospital, prior to the COVID-19 pandemic onset, was conducting a Phase II double blinded placebo controlled clinical trial in subjects with long standing diabetes and all who were adults at the time of enrollment. Subjects had “established” diabetes with more than 10 years of type 1 diabetes. These patients also had other co-morbid conditions such as hypertension, older age, cardiovascular disease, CNS disease i.e., strokes, kidney disease, prior lung disease, hypercholesteremia, cancer, asthma, etc. Prior to February 2020, all 150 subjects had been randomized to BCG (100) or placebo (50). All subject prior to February 2020 had already received 2 BCG vaccines (2 in year 01; 1 vaccine in year 02) prior to the pandemic. The FDA was asked permission to only unblind this data as it relates to COVID infection, outcome, of symptoms not the primary outcome of HbA1c. The COVID study was also designed for an additional family cohort comparisons as well as comparisons of BCG treated versus untreated. This COVID-19 study is unique in testing whether multi-dose BCG can protect adults from a pandemic. This trial is unique in that the subjects were at high risk for mortality and morbidity from COVID-19 infections.

These are the preliminary results comparing BCG to Placebo subjects for COVID symptoms:

Using any level of anti-COVID antibody detection as a sign of COVID exposure, symptoms were scored:

BCG Treatment Group with Anti-Covid Antibodies; 18% with New Covid Antibodies
Symptom free: 15%
Mild symptoms lasting a short time: 3%
Severe symptoms: 0%
   Placebo Group with Anti-COVID Antibodies: 24% with New Covid Antibodies
Symptom free: 14%
Mild symptoms lasting a short time: 0%
Severe symptoms: 10%

This double blinded placebo controlled and randomized data clearly shows multi-dose BCG vaccinations in naive US citizens with no prior BCG vaccine, a protection from severe symptoms (p<0.02) and also in some cohorts total protection from the infection as defined by symptoms (p=0.06). Data is in real time and being monitored by a separate statistical group as the exposures to COVID-19 continue in a population with a serious co-morbid disease. Case studies of COVID exposed diabetes patients are also being collected and document how close contacts and family members, not treated with BCG, prior to the COVID exposures were affected by COVID.

### Example 2

We are also conducting a family cohort study with monthly surveys of any COVID symptoms in cohort enrolled in the trial and family members. All are screened for antibodies and scored for the severity of their symptoms. The preliminary results comparing BCG to Placebo subjects for COVID symptoms compared to relatives also with COVID symptoms/antibodies are as follows:

BCG Treated Group with COVID Antibodies (n=6)

| 0 symptoms | 2 symptoms | 3 symptoms |
|---|---|---|
| n = 3 (50%) | n = 3 (50%) | n = 0 (0%) |

Percentage of relatives with COVID antibodies: 100%

Family Members with COVID Antibodies (n=11)

| 0 symptoms | 2 symptoms | 3 symptoms |
|---|---|---|
| n = 1 (9%) | n = 1(9%) | n = 9 (82%) |

Placebo Treated Group with COVID Antibodies (n=1)

| 0 symptoms | 2 symptoms | 3 symptoms |
|---|---|---|
| n = 0 | n = 0 | n = 2 (100%) |

Percentage of relatives with COVID antibodies: 100%

Family Members with COVID Antibodies (n=1)

| 0 symptoms | 2 symptoms | 3 symptoms |
|---|---|---|
| n = 0 | n = 0 | n = 4 (100%) |

Scoring is 0=no symptoms; 2=mild symptoms; 3=severe symptoms
All cohorts with anti-COVID antibodies and relatives in same household

The data shows no death, no severe symptoms nor ICU admissions, for the COVID infection diabetics who had received multi-dose BCG therapy compared to the placebo group. Therefore, with a co-morbid disease, multi-dosing of BCG prevents COVID symptoms and or severe disease progression. In the double blinded placebo controlled trial the data statistically shows protection from COVID if vaccinated with BCG and this results in no to mild symptoms.

In the family study, 100% of samples family members are similarly infected with COVID based on the presence of antibodies. Again, the BCG treated diabetic subjects have no or few symptoms and family members have most commonly severe symptoms. Therefore, in families, BCG protects only the BCG immunized cohort from disease. The data shows COVID spreads within household in this cohort is with 100% infectivity. The data shows BCG offers unique protection from severe COVID symptoms; severe symptoms are only a trait of only non-vaccinated family members. The data shows BCG vaccinated subjects often have no symptoms in 50% of the cases and 50% of the time mild symptoms. Family members have no symptoms 9% of the time, mild symptoms 9% of the time and severe symptoms 82% of the time.

All subjects enrolled in this double blinded placebo controlled trials have co-morbid disease and are all adults with long standing diabetes. Many subjects also have hypertension, hypercholesteremia, and some have developed complications of their longstanding diabetes of greater than 10 years in duration.

### Example 3

We also tested whether repeat BCG vaccinations protected from other viral infectious diseases. We used the protein array technology of different fragments of the COVID virus capsid protein to profile all changes in existing antibodies to diverse infectious diseases. This technology detects antibodies in blood that indicate a past infection from viruses. It also tests whether those antibody levels may change after BCG vaccinations. A boost in antibodies to past infections after BCG would indicate the person now has enhanced immunity to many different infections, even decades in the past without “re-seeing” the infection.

From the serum of BCG vaccinated diabetic patients, we investigated over a 3 year time course if the BCG boosted the immune system to make more antibodies possible enhanced immunity to infections.

This data evidenced the slope of antibody levels to diverse human viruses uniformly increased over the 3 years of monitoring from the first BCG vaccination. This is the first data to show that repeated BCG vaccinations can protect from diverse viral infections, and that it does this by boosting the level of diverse serum antibodies from previous exposures. The taxon species studied included Human rhinovirus A serotype 89, Coxsackievirus A21, Human rhinovirus 3, Torque teno midi virus 1, Human rhinovirus 16, Coxsackievirus A24, Poliovirus type 2, Coxsackievirus B5, Enterovirus B, Human rhinovirus 2, Echovirus 9, Coxsackievirus B6, Coxsackievirus A13, Echovirus 11, Human rhinovirus 1B, Echovirus 30, Human rhinovirus A39, Human rhinovirus 14, Echovirus 16, Bovine respiratory syncytial virus, Human rhinovirus 23, Tick-borne Powassan virus, Simian adenovirus E22, Poliovirus type 3, Coxsackievirus B3, Rhinovirus B, Coxsackievirus B4, Echovirus 5, Feline coronavirus, Hepatitis C virus genotype 3a, Echovirus E2, Human rhinovirus 1A, Simian adenovirus 24, Mamastrovirus 1, Poliovirus type 1, Hepatitis B virus genotype E subtype ayw4, Echovirus 1, Coxsackievirus B2, Human astrovirus-8, Human papillomavirus type 38b, Human adenovirus 62, Human enterovirus 70, Adeno-associated virus-3, New York virus, Human hepatitis A virus genotype IIA, Coxsackievirus A9, Rift valley fever virus, Human adenovirus D serotype 15/H9, Hepatitis C virus genotype 2k, Southampton virus, Human astrovirus-3, and Echovirus 12.

### Example 4

In a randomized, double-blinded, placebo-controlled trial, we evaluated the efficacy of the >100-year-old Bacillus Calmette-Guerin (BCG) vaccine for prevention of Covid-19 in an unvaccinated, at-risk US population. Our trial was adapted from an ongoing trial of multi-dose BCG vaccination for another indication. That trial was completely enrolled and BCG dosed before the start of the Covid-19 pandemic, January 2020. Using a parallel trial design, we added co-primary outcomes: efficacy of BCG in prevention of symptomatic and molecularly confirmed Covid-19; and impact of BCG on a possible reduction of all infectious disease including Covid-19 symptoms and severity.

In this parallel trial design, 144 at-risk subjects were enrolled, 96 were randomized to multi-dose BCG and 48 to placebo with 2:1 randomization. Over a 15-month time period, a cumulative incidence of 12.5% of placebo-treated (6/48) and 1% of BCG-treated participants (1/96) met study criteria for confirmed Covid-19, yielding a vaccine effectiveness of 92% (0.99 posterior probability, p=0.009). The BCG group displayed fewer Covid-19 and overall infectious disease symptoms and lesser severity as captured in the Total or Average Infectious Disease Index (each p=0.04) and fewer infectious disease events per patient (p=0.004). No BCG-related systemic adverse events occurred.

As is described herein, multi-dose BCG vaccinations are safe and highly effective against symptomatic Covid-19. BCG's broad-based infection protection accordingly provides platform protection against new SARS-COV-2 variants and other pathogens.

### Methods

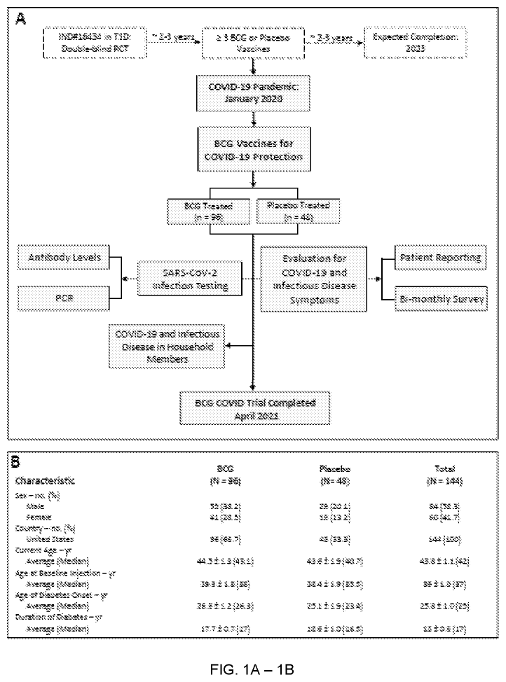

The following are the methods we used in Example 4 to assess the impact of multi-dose BCG vaccinations for protection from SARS-COV-2 infections and other infections (FIG. 1A).

### Trial Objectives, Participants and Oversight

We assessed the efficacy of ≥3 BCG vaccinations (Tokyo-172 strain) versus placebo in a randomized double-blinded clinical trial. The trial duration was 15 months, starting at the beginning of the SARS-COV-2 pandemic in the US (01/01/2020) and ending with the U.S. launch of Covid-19 vaccines in April 2021 (FIG. 1A). Clinical trial participants were 18-50 years old with a co-morbid condition, type 1 diabetes (FIG. 1B). The current trial was a parallel study using already enrolled double-blinded participants in a multi-dose BCG vaccine trial assessing its 5-year impact on blood sugar control. At the three-year time point of the original study, this parallel study on Covid-19 protection was initiated, while the original trial continued in a double-blinded fashion.

A total of 144 subjects were enrolled, with 96 receiving BCG and 48 receiving placebo vaccinations. There was no patient attrition over the 15-month course. The original exclusion criteria included positive purified protein derivative (PPD) test, positive T-spot test for tuberculosis, or born in a foreign country with mandatory BCG vaccinations. The goal of these exclusion criteria was to prevent prior ongoing and durable protection from Mycobacterium bovis (the origin of the BCG vaccine) or Mycobacterium tuberculosis (TB) exposures causing long-term protection. Exclusion criteria also included no active glucocorticoids treatment, chronic immunosuppressive medications, or currently living with an immunosuppressed individual to prevent an adverse event from the administration of this live vaccine. All subjects lived within the United States.

This trial had three types of oversight. At 6-month intervals, this trial had audits from either Massachusetts General Brigham (MGB) Division of Quality Management or from outside auditors (Advanced Clinical Trials, Deerfield, IL). All trial data was processed by two unblinded statisticians, and an independent data and safety monitoring board (DSMB) met at 6-month intervals to monitor subject safety and reporting compliance.

### Trial Procedures

The BCG vaccine or saline placebo (both 0.1 ml volume per dose) was administered intradermally. Site staff were responsible for reporting all drug- and non-drug-related safety information and were unaware of group assignments. A separate study nurse, at 4 weeks after each vaccine dose, evaluated the local vaccine administration site.

### Clinical and Laboratory Testing

Monitoring over the 15-month surveillance period included a questionnaire of possible infectious disease symptoms including Covid-19 symptoms and was administered during each 6-month clinic visit as well as blood draws. The infectious disease survey was also an emailed survey and completed bimonthly by trial participants for themselves and for any adult household member with infectious symptoms. Subjects would also call the clinic to report infectious symptoms for themselves or their family members. Participants also directly reached out to the clinic to report infections. Blood from household members for confirmation of SARS-COV-2 infection was locally obtained (Quest Diagnostics, Secaucus, NJ). The symptoms questionnaire for study participants and infected household members followed FDA

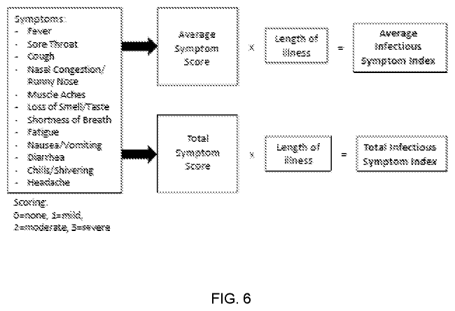

Guidance for Industry (FDA Guidance for Industry, “Assessing COVID-19 related symptoms in outpatient adult and adolescent subjects in clinical trials of drug and biological products for COVID-19 prevention or treatment” September 2020). For each symptom, the participants provided a severity score of 0 (none), 1 (mild), 2 (moderate) or 3 (severe). The length of the infectious illness in days was also reported. From the individual symptom scores a Total Symptom Score and an Average Symptom Score were tabulated for infectious disease symptoms. Then, multiplying by the length of illness, the Total Infectious Symptom Index or the Average Infectious Symptom Index was assessed (FIG. 6).

Covid-19 is defined according to the Food and Drug Administration (FDA) by the presence of at least one of 12 symptoms (FDA Guidance for Industry, September 2020) (headache, chills/shivering, diarrhea, nausea/vomiting, fatigue, shortness of breath, loss of smell or taste, muscle aches, nasal congestion, cough, sore throat and fever) and molecular confirmation. These symptoms, other than the loss or smell and taste, are symptoms of most infectious diseases, so the survey was used for both purposes. The criteria for confirmed Covid-19 used in this trial was to report at least one symptom of Covid-19 and to test positive in 5 or more out of 10 Covid-19 molecular diagnostic tests. These tests either detected the presence of virus by serology for the presence of anti-COVID-19 antibodies or in some cases were Point of Care testing using available methods. A summary of all testing methods is presented (Table 1).

TABLE 1
COVID-19 Detection Methods

| | | |
|---|---|---|
| I-IX. COVID-19 Directed | | Antibody Binding Regions |
| Antibodies | | |
| I-VIII. IgG Antibodies | Protein | SARS-CoV-2 Amino Acid (AA) |
| | array | regions** |
| I. Spike 1 | | 115-QSLLIVNNATNV-126 |
| II. Spike 1 | | 145-YHKNNKSWMESE-156 |
| III. Spike 1 | | 307-TVEKGIYQTSNF-318 |
| IV. Spike 1 | | 319-RVQPTESIVRFP-330 |
| V. Spike 1 | | 457-RKSNLKPFERDI-468 |
| VI. Spike 2 | | 710-NSIAIPTNFTIS-721 |
| VII. Spike 2 | | 1088-HFPREGVFVSNG-1099 |
| VIII. RNA Polymerase | | RNA polymerase |
| IX. IgG Antibodies | ELISA | Spike protein* |
| X. Point of Care | | |
| Nasal Swab | RT-PCR | Nucleocapsid RNA |
| Rapid Antigen Test | Antigen | Majority nucleocapsid protein |
| | Test | |
| Antibody Test | ELISA | Nucleocapsid or Spike |
| | | antibodies |

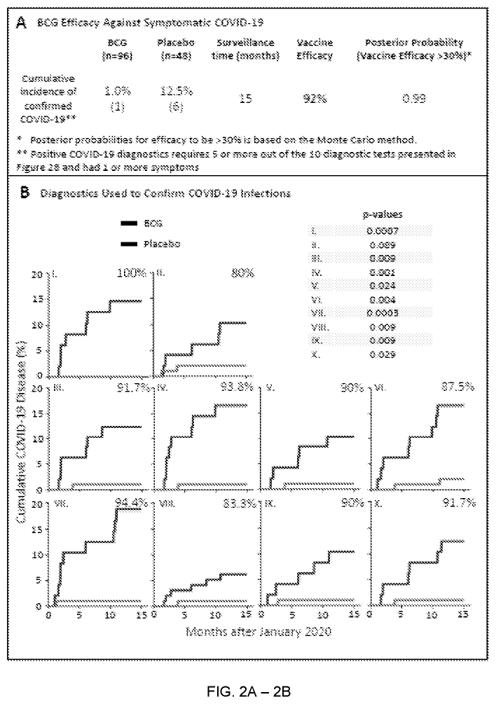

*SARS-CoV-2 Protein Microarray: 2-in-1 protein and peptide assay from CDI Labs, Baltimore, Maryland (CDICOV2-001.0)
**AntiCoV-ID ™ IgG ELISA from Akston BioSciences, Beverly, Massachusetts (SKU: 600016)
I-X represent the same numbers and thus diagnostic tests shown in FIG. 2

Table 1 provides an overview of all the analytical methods for Covid-19 detection that are described in this Example (FIG. 2). For the confirmation of SARS2 infections the presence of subject antibodies to the SARS2 virus were sought and confirmed through multiple methods (I-VIII). For SARS2 antibodies detected, the average and standard deviation of antibody levels prior to the onset of Covid-19 pandemic (Pre-2020) was determined. The average of the levels after the start of the pandemic (2020 and 2021 data) was also calculated. Using these averages and the Pre-2020 standard deviation the Z-score per patient was then calculated. The Z-score thus represents the difference in average Pre-Covid and average Covid signal levels, expressed as the number of standard deviations of pre-Covid. A Z-score of ≥3 was considered to represent a statistically significant difference. Efficacy was calculated from % patients in BCG and placebo groups that had a Z-score ≥3 using the formula: (p1−p2)/p1×100, where p1 is the % Covid-positive in the placebo group and p2 is the % Covid-positive in the BCG group. We also used an ELISA assay specific to antibodies against the Receptor Binding Domain (RBD) portion of the S1 spike subunit (IX). Patients in their community locations also received a diagnosis of Covid-19 infections through a variety of methods (X).

Since this study started at the beginning of the pandemic when Point of Care testing was difficult to obtain, often dependent on a narrow time window for sample collection (PCR) and highly variable methods, serology for Covid-19 antibodies was essential for the Covid-19 diagnosis. A subject was deemed positive for a particular Covid-19 antibody assay if the serology antibody Z-score was ≥3, i.e., at least 3 standard deviations higher than during the pre-Covid period (baseline). Serum was obtained in all cases within 3 months of the infection for the detection of IgG antibodies.

### Efficacy

This trial had the following primary endpoints on potential benefits of multi-dose BCG: 1) efficacy against molecularly confirmed Covid-19 with possible protection from Covid-19 or infectious disease severity and to determine if multi-dose BCG can protect from other infectious diseases (Table 2).

TABLE 2
Study Synopsis

| | |
|---|---|
| Title | USE OF BCG-JAPAN VACCINATIONS TO |
| | PREVENT COVID-19 AND LIMIT |
| | SYMPTOMS IN AT RISK SUBJECTS |
| Short Title | BCG to Prevent COVID-19 |
| Clinical Phase | Phase III |
| Number of Sites | 1 enrollment site; US based recruitment |
| IND Sponsor | Massachusetts General Hospital, Boston, MA |
| Study Objectives | To determine if multi-dosing BCG-Japan |
| | (Tokyo-172) can protect high risk subjects from |
| | COVID-19 symptomatic infection. |
| | To determine the impact (severity, duration of |
| | symptoms, absence from work) of BCG-Japan on |
| | Covid-19 symptoms. |
| | To determine if BCG-Japan can also protect from |
| | other infectious diseases. |
| Study Design | Randomized, double blind, placebo-controlled |
| | clinical trial of three doses of BCG-Japan |
| Co-primary Outcomes | 1. Prevention of Covid-19 antibody conversion of |
| | symptomatic COVID-19 disease |
| | 2. Prevention of infections that are not |
| | COVID-19 |

### Statistical Analysis

Vaccine efficacy is defined by (p1−p2)/p1×100, where p1 is the % Covid-positive in the placebo group and p2 is the % Covid-positive in the BCG group. The posterior probability that the vaccine efficacy is greater than 30% was calculated with the use of a Bayesian beta binomial model using WinBUGS (Lunn et al., Statistics and Computing, 10:325-37, 2000). Further details are provided herein. Average antibody levels were compared using Student's T-testing in Prism (Graphpad Software, San Diego, CA) or Microsoft Excel. The number of patients positive in the BCG versus the Placebo cohort were compared using Fisher's Exact Test (See, for example GraphPad, Analyzing a 2×2 contingency table at graphpad.com). Differences in the symptoms scores between participants and household members were compared between the BCG treated group and the Placebo treated group using a Two-Sample Wilcoxson test. Statistics were considered significant at p<0.05.

### Results

Results for Example 4 were as follows.

### Trial Population

We assessed the impact of multi-dose BCG vaccinations for protection from SARS-COV-2 infections and other infections (FIG. 1A). This consort diagram represents all enrolled participants from Jan. 1, 2020 to April 2021 for this double-blinded randomized clinical trial testing repeat Tokyo-172 BCG vaccination vs placebo. All 144 subjects were followed for 15 months with a 2:1 randomization and no drop-outs. Data collection for this trial ended on April/2021, the date when subjects started to receive provisionally approved Covid-19 specific vaccines. All 144 participants were US citizens not previously vaccinated with BCG at birth and had long-standing type 1 diabetes, a co-morbid condition for worse Covid-19 symptoms (FIG. 1B) (Barrett et al., Intensive Care Unit Admission, Mechanical Ventilation, and Mortality Among Patients With Type 1 Diabetes Hospitalized for COVID-19 in the U.S., Diabetes Care, 2021).

### Safety

There were no BCG-related systemic adverse events in any of the participants. BCG vaccines do cause local reactogenicity that usually appears at 2-4 weeks. No excess local reactions defined as an injection site reaction of >2 cm were reported.

### Covid-19 Incidence

Confirmed cases of Covid-19 were defined as exhibiting one or more FDA-defined symptoms and 5 or more positive results out of 10 diagnostic tests. Using these criteria, the cumulative incidence of confirmed Covid-19 in the BCG cohort was 1 out of 96 (1%), whereas it was 6 out of 48 (12.5%) in the placebo group. The efficacy of BCG to prevent Covid-19 infections was 92% (FIG. 2A). Monte Carlo statistics resulted in a Posterior Probability (with Vaccine Efficacy >30%) of 0.99. FIG. 2A shows the cumulative incidence of confirmed Covid-19 as a primary endpoint of this 15-month clinical trial. Vaccine efficacy was defined as (p1−p2)/p1×100 where p1 is the percentage Covid-positive subjects in the placebo group (12.5%) and p2 is the percentage Covid-positive subjects in the BCG group (1%). The criteria for confirmed Covid-19 were as follows: reporting symptom(s) of Covid-19, as well as testing positive for 5 or more out of 10 in vitro diagnostic tests. Cumulative findings from these 10 diagnostic tests are shown in FIG. 2B. During the 15-month surveillance time, one BCG recipient out of 96 (1.0%) met our criteria for confirmed Covid-19. In contrast, 6 out of 48 placebo recipients met the criteria (12.5%). Fisher's Exact testing showed a significant difference (two-tailed p=0.006). Calculated vaccine efficacy was 92% and the posterior probability (vaccine efficacy >30%) was 0.99. This was calculated using the Monte Carlo method. The diagnostic test details are represented in Table 1.

Graphs of cumulative Covid-19 for all 10 molecular diagnostics were remarkably consistent for disease detection and vaccine efficacy calculated from different methods (FIG. 2BII-X; listed in Table 1). In all molecular assays monitoring Covid-19 infections, there was a higher percentage of infected patients in the placebo group compared to the BCG group. Vaccine efficacies (upper right corner of each graph) for individual assays ranged from 80% to 100% and, with the exception of II, all were statistically significant (FIG. 2B).

FIG. 2B shows the 10 in-vitro diagnostic tests used to diagnose Covid-19 (along with positive symptoms). These included the presence of Covid-19-specific antibodies to various SARS-COV-2 virus epitopes through protein display (I-VIII), antibodies to the Spike protein with an ELISA test (IX) and Point of Care testing (X). Thus trial required at least 5 of 10 detection methods to be positive, along with symptoms, for having confirmed Covid-19. For the antibody assays, a patient was considered positive when the test resulted in a Z-score of ≥3. In the cumulative graphs, the x-axis data shows the time-period of the 15-month trial. The Y-axis shows the cumulative percentage of positive subjects. Except for the point of care graph (X), all other graphs represent the percentage of BCG and placebo patients with a Z-score of ≥3 for the anti-SARS-COV-2 antibody binding to a given protein region of the virus, i.e. the average antibody level during the Covid trial period was at least 3 standard deviations greater than the average level in the period preceding the Covid trial (baseline). The percentiles at the top-right of each graph represent the calculated vaccine efficacy if this test alone were used to diagnose Covid-19 disease. Respective efficacy and Fisher's Exact p values for each Covid-19 antibody test were as follows: (I) 100%, p=0007; (II) 80%, p=0.089; (III) 91.7%, p=0.009; (IV) 93.8%, p=0.001; (V) 90%, p=0.024; (VI) 87.5%, p=0.004; (VII) 94.4%, p=0.0003; (VIII) 83.3%, p=0.009, (IX) 91.7%, p=0.029; (X) 90%, p=0.009; Table 1 shows the viral protein regions for each anti-Covid antibody tested.

### BCG Reduces Overall Infectious Disease Susceptibility

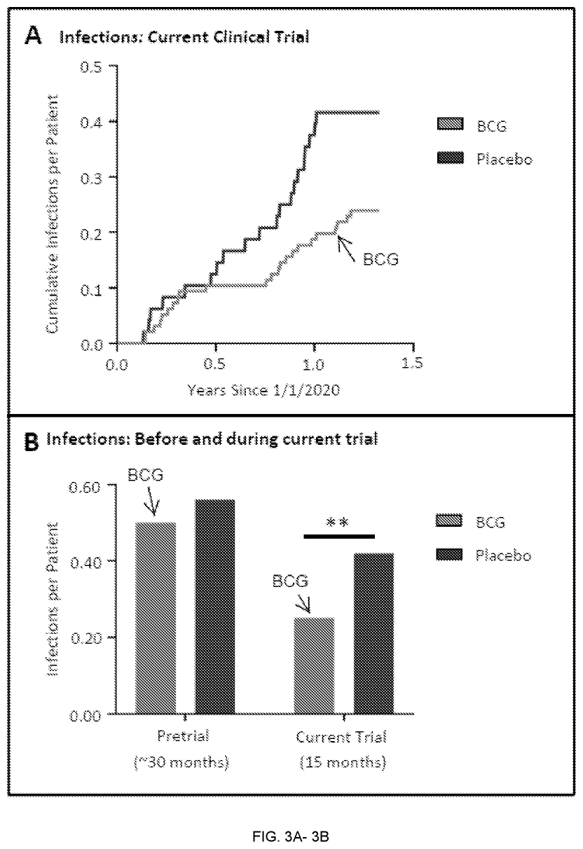

We analyzed infectious disease events collected as “adverse events” by the Medical Dictionary for Regulatory Activities (MedDRA) classification coding (FIG. 3, Table 3). Using the time period of the current Covid-19 trial, we analyzed infectious diseases including Covid-19 and other infections. The cumulative infectious diseases reported by the subjects and represented a cumulative infections per patient were significantly lower in the BCG vs placebo group (Poisson model comparing adverse events rates p=0.004; FIG. 3A, Table 3B). These findings are likely to represent the minimum protection.

To look at this time period of BCG onset of vaccine effectiveness but in the setting of infection protection, the clinical trial infectious disease adverse events were broken into the pre-Covid trial period itself ((Table 3A) during which the third BCG vaccine was administered versus the 15-month time period of this current clinical trial (FIG. 3B, Table 3B)). While comparison of infectious adverse events in BCG versus placebo groups during the current trial had a significant Poisson distribution (p=0.004), there was no significant difference during the Pre-Covid trial period (Poisson p=0.46). These data indicate that it might take about 2-3 years after the first vaccine for maximal effectiveness for the platform infectious disease protection.

TABLE 3A

| MedDRA | | Adverse Events per Patient: | |
|---|---|---|---|
| Code: | Adverse Event | BCG | Placebo |
| 10006451 | Bronchitis | 0.02 | 0.02 |
| 10010106 | Common cold | 0.06 | 0.06 |
| 10011224 | Cough | 0.01 | 0.02 |
| 10014011 | Ear infection | 0.09 | 0.04 |
| 10021789 | Infection | 0.03 | 0.06 |
| 10034839 | Strep throat | 0.02 | 0.04 |
| 10040745 | Sinus infection | 0.21 | 0.21 |
| 10046571 | Urinary tract infection | 0.04 | 0.08 |
| 10062352 | Upper respiratory infection | 0.01 | 0.02 |
| | Total: | 0.50 | 0.56 |

| | |
|---|---|
| Poisson distribution p | 0.46 |

TABLE 3B

| MedDRA | | Adverse Events per Patient: | |
|---|---|---|---|
| Code: | Adverse Event | BCG | Placebo |
| 10016791 | Flu-like symptoms | 0.18 | 0.19 |
| 10040745 | Sinus infection | 0.02 | 0.04 |
| 10046571 | Urinary tract infection | 0.02 | 0.06 |
| 10084268 | COVID-19 | 0.03 | 0.13 |
| | Total: | 0.25 | 0.42 |

| | |
|---|---|
| Poisson distribution p | 0.004 |

Tables 3A and 3B show a listing of analyzed infections in the pretrial and trial time periods. Infections were documented (through adverse events (AEs) reporting and surveys) during the 2½ year Pre-Trial period (Table 3A) and during the 15-month Trial period (Table 3B). Listed are only the infections for which multiple events were documented in both BCG and placebo groups. Poisson distribution analysis shows that there was no significant difference between BCG and placebo AEs during the Pre-Trial Period (p=0.46), whereas during the Trial period the difference was significant (p=0.004).

The Pretrial time period was when all clinical trial subjects received their ≥3 BCG vaccines or placebo vaccines; the Current Trial was when the subjects were monitored during the 15-month observation period of this clinical trial during the Covid-19 pandemic. The lack of a statistical difference in infectious diseases between BCG and placebo groups in the pre-trial period suggests the 2½-year period with the BCG vaccinations underway might not yet fully protect against infectious disease and thus a longer length of time is necessary for maximal infectious disease protection. It appears by the time this parallel clinical trial started the prior BCG vaccinations were protecting from Covid-19 and all infections. **p<0.01

### Infectious Disease Symptoms and Severity

Our intent was to evaluate the severity of Covid-19 symptoms with BCG treatment as compared to placebo in confirmed Covid-19-positive patients, but this was not possible because only one subject in the BCG group fit our criteria for confirmed Covid-19. We therefore analyzed the symptoms in symptomatic BCG- and placebo-treated participants, regardless of whether they were confirmed Covid-positive to understand the impact of BCG on overall infectious disease severity.

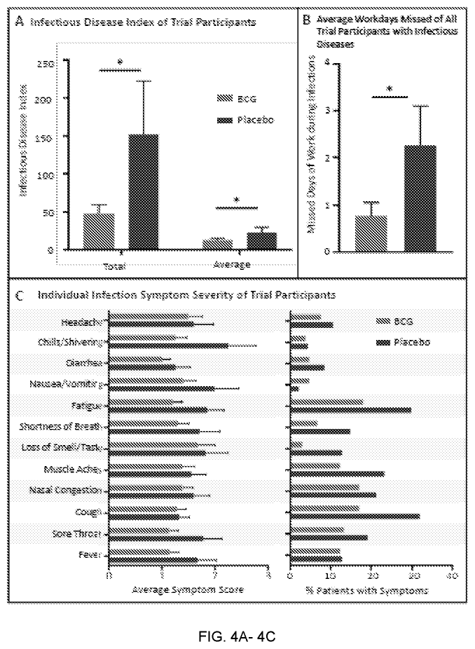

Using data from symptom surveys based on FDA guidelines that were completed bimonthly, we calculated a Total and Average Infectious Disease Index (FIG. 6). Indexes for each individual patient were calculated separately and then averaged across all subjects in the BCG and placebo cohorts (FIG. 4A). Comparing only symptomatic patients, the Total Covid Index for infectious diseases in the BCG cohort (48±11, n=31) was significantly reduced vs placebo (152±70, n=20; p=0.04). The Average Infectious Disease Index also showed a significant decrease (BCG 13±2, n=31, versus placebo 23±7, n=20; p=0.04). This indicates that BCG vaccination reduced the severity and duration of all infectious disease symptoms as compared to placebo. Symptomatic patients in the BCG group also reported a significantly lower number of days of missed work as compared to in the placebo group (FIG. 4B; BCG 0.77±0.28; placebo 2.26±0.84, p=0.02). All individual average symptom scores (12 out of 12) were more severe in the placebo group vs the BCG group (FIG. 4C Left column). For 11 out of 12 symptoms there also was a higher percentage of patients in the placebo group versus BCG group (FIG. 4C Right column).

### Infection Severity in Trial Participants as Compared to Household Members

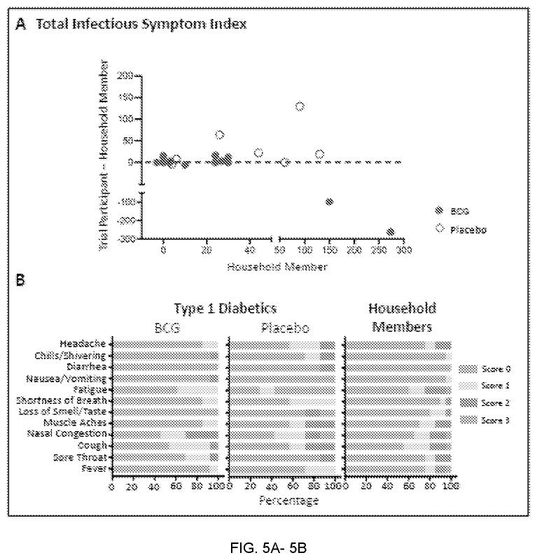

We also studied the infection disease severity of trial participants compared to non-diabetic adults living in the same household. We collected infectious disease symptom information for trial participants and for cohabitating adult partners in 20 households (13 BCG and 7 placebo) and determined the differences in Total Infectious Symptom Index for each household (FIG. 5A, FIG. 6). The figures show BCG recipients had comparable or milder symptoms as compared to their household members, whereas most Placebo recipients had more severe disease as compared to their household members. The symptom scores between participants and household members were compared between the BCG treated group and the Placebo treated group using a Two-Sample Wilcoxson test (p=0.049, 2-tailed). The stacked horizontal bars in FIG. 5B show the distributions of the infectious disease symptom scores in each group. BCG recipients had milder symptoms as compared to Placebo recipients and even as compared to non-diabetic household member controls.

The distribution of symptoms in FIG. 5B show once again that for BCG-treated trial participants individual symptoms were minimized with vaccinations compared to placebo. *p<0.05 (1-tail, unpaired).

This randomized double-blinded placebo-controlled trial showed that repeat BCG vaccination is safe and prevents Covid-19 with an efficacy of 92% and lessens all infectious disease events and symptomatology. BCG-vaccinated adults also had reduced incidence of all infections.

This clinical trial has several strengths and limitations. First, this trial is, to our knowledge, the first peer-reviewed randomized double-blinded trial of multi-dose BCG for Covid-19 protection and broad-based infection protection. A single dose of BCG with immediate Covid-19 disease monitoring showed neither protection from Covid-19 infections nor protection from disease severity (Giamarellos-Bourboulis et al., Cell, 183:315-23 e9, 2020). Second, the trial uses a very potent strain of BCG, Tokyo-172. BCG strain differences for other off-target indications are important and this strain of BCG exhibits some of the highest in vitro potency and is highly immunogenic. Third, the study population is high-risk, suggesting that the vaccine is efficacious in a susceptible population. Fourth, the study uses rigorous molecular methods to confirm current or past infection. Fifth, the findings should help overcome vaccine hesitancy because of BCG's 100-year strong safety record and its international use as a neonatal vaccine. Finally, subjects are from the United States. This is important because all subjects, prior to enrollment, were confirmed by diagnostics and by history to be unexposed to tuberculosis and lacking prior BCG vaccinations. The United States has never had a country policy of neonatal BCG vaccinations.

The most important limitation of this trial is that it may not have been long enough to capture the full protectiveness of BCG for Covid-19 and more infectious diseases. BCG does not work as fast as other Covid-19 vaccines likely offers long-term effects perhaps decades-long.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the claims.

1. A method for the prophylactic treatment of a coronavirus infection in a subject comprising administering at least two doses of a Bacillus Calmette-Guerin (BCG) vaccine to a type I diabetic.

2. The method according to claim 1, wherein the coronavirus is SARS COV-2.

3. The method according to claim 1, wherein the type I diabetic has not been previously vaccinated with a BCG vaccine.

4. The method according to claim 1, wherein the volume of the dose is delivered in about a 0.1 ml volume and the BCG is about 2.09 to 50×10⁶ cfu per 0.5 mg BCG/10 doses.

5. The method according to claim 1, wherein the dose is administered intradermally or percutaneously.

6. The method according to claim 1, wherein the type I diabetic is age 18 or older.

7. The method according to claim 1, wherein the type I diabetic is age 12 to 17.

8. The method according to claim 1, wherein the type I diabetic is 11 months to age 11.

9. The method according to claim 1, wherein the type I diabetic received three doses of BCG vaccine.

10. The method according to claim 1, wherein the type I diabetic received greater than three doses of BCG vaccine.

11. The method according to claim 1, wherein the type I diabetic received BCG (Tokyo-172 strain) vaccine.

12. The method according to claim 1, wherein the type I diabetic receives two doses of BCG vaccine four weeks apart.

13. The method according to claim 12, wherein the type I diabetic receives a booster vaccine dose.

14. The method according to claim 1, wherein the type I diabetic is an established type 1 diabetic.

15. The method of claim 1, wherein the type I diabetic has a co-morbidity.
